(19) United States
(12) Patent Application Publication
(10) Pub. No.: US 2023/0338673 A1
(43) Pub. Date: Oct. 26, 2023

(54) NEBULIZER AND NEBULIZER SYSTEM

(71) Applicant: Pulmotree Medical GmbH, Munich (DE)

(72) Inventors: Pia GÖRSTNER, Munich (DE); Kristian KELLNER, Munich (DE); Ulf KRÜGER, Munich (DE)

(73) Assignee: Pulmotree Medical GmbH, Munich (DE)

(21) Appl. No.: 18/025,468

(22) PCT Filed: Aug. 20, 2021
(86) PCT No.: PCT/EP2021/073208

§ 371 Date: Mar. 9, 2023

(30) Foreign Application Priority Data
Sep. 9, 2020 (EP) 20195298.3

(51) Int. Cl. A61M 11/00

(52) U.S. Cl. CPC A61M 11/005; A61M 2205/60

(57) ABSTRACT

The application is directed to a mesh nebulizer and related system including a dosing unit for providing a predetermined amount of a therapeutic agent, wherein the therapeutic agent is delivered in a portion container to be opened by pressure. The dosing unit comprises an uptake chamber with a dosing opening for dispensing the therapeutic agent, an opening for inserting a portion container and a locking body for the opening. The locking body is adapted to be moved from an open position in which the locking body releases the opening to a closed position in which the locking body closes the opening. The locking body is further adapted to be moved from the closed position to a drainage position in which the locking body applies pressure to the portion container to open the portion container and release the therapeutic agent.

The invention relates to a nebulizer and a nebulizer system.

Nebulizers are medical devices used to deliver liquid therapeutic agents into a patient's respiratory tract. For this purpose, common nebulizers comprise a nebulizer head in which the therapeutic agent is nebulized and added to an air stream inhaled by the patient. Different types of nebulizer heads are known, which generally function according to one of the following principles:
Jet nebulizer: In this type of nebulizer head, a pressurized gas is passed through a reservoir of the therapeutic agent via a nozzle, which atomizes the therapeutic agent into a mist. However, such nebulizer heads are bulky and noisy, so that they are now rarely used.
Ultrasonic nebulizer: In this type of nebulizer heads, the therapeutic agent is passed over an ultrasonic vibrator, which atomizes the therapeutic agent into a mist.
Mesh nebulizer: In this type of nebulizer heads, the therapeutic agent is atomized by an ultrasonic mesh that covers the liquid surface of the therapeutic agent.

Furthermore, nebulizers comprise a dosing unit by means of which a predetermined or predeterminable amount of a therapeutic agent is supplied to the nebulizer head. The following principles are known:
Open dosing: The dosing unit can be designed as an open dosing unit to which the therapeutic agent is fed manually from portion ampoules or dosing pipettes. In this case, handling is usually complicated and there is also a risk of incorrect dosing. Incorrect dosing can be caused by incomplete emptying of portion ampoules (underdosing) or by incorrect operation of a dosing pipette (overdosing or underdosing).
Closed dosing: In the case of closed dosing units, portion ampoules with the therapeutic agent are usually inserted into the dosing unit, where they are lanced open and emptied by a mechanism. Even with known closed dosing units, the handling is still not optimal. In addition, the use of single-portion ampoules (for both open and closed dosing units) produces considerable amounts of packaging waste.

Both the nebulizer head and the dosing unit of nebulizers can only be used for a limited period of time, as their functional reliability is reduced by wear and/or dirt. In particular, dirt deposits, e.g. from the supplied breathing air flow, can form a breeding ground for pathogens in combination with moisture. Currently available nebulizer heads and dosing units cannot be cleaned easily or sufficiently thoroughly. In addition, the general performance of dosing units and nebulizer heads decreases over time due to wear.

Therefore, the respective components have to be replaced regularly. Therefore, there are nebulizer systems that comprise a base unit and several nebulizer heads and/or dosing units that can be used together with the base unit.

Nebulizers further comprise a control unit that controls the nebulizer head and/or the dosing unit. The control unit is usually located in the base unit and has one or more sensors that determine, for example, the speed of an air flow through the nebulizer head.

The control unit can then activate the nebulizer head when there is sufficient air flow to atomize the therapeutic agent.

Some nebulizers have a communication interface to transmit measured values from the sensors to an external control or visualization device. These measurement values can then be used to provide feedback to the patient about their usage behavior and possible improvements.

In general, there is a need to provide nebulizers with improved handling, function and/or functional safety.

According to a first aspect, this object is solved by a nebulizer comprising a dosing unit for providing a predetermined amount of a therapeutic agent, wherein the therapeutic agent is delivered in a portion container to be opened by pressure; wherein the dosing unit comprises an uptake chamber with a dosing opening for dispensing the therapeutic agent, an opening for inserting a portion container and a locking body for the opening; wherein the locking body is adapted to be moved from an open position in which the locking body releases the opening to a closed position in which the locking body closes the opening; and wherein the locking body is further adapted to be moved from the closed position to a drainage position in which the locking body applies pressure to the portion container to open the portion container and release the therapeutic agent.

For example, the portion container may have a top cover, a bottom cover, and a closure seam connecting the top cover to the bottom cover. To release the therapeutic agent, the portion container can be torn open by pressure along the closure seam so that the therapeutic agent can escape. The portion container can also tear along only part of the sealing seam, or at other points of the upper and lower shell. If necessary, tear lines can be introduced into the respective material sections.

The movement of the locking body from the open position to the closed position may be or comprise a linear movement. For example, the locking body can be a cap that is placed on the dosing unit from above.

The movement of the locking body from the open position to the closed position may be or comprise a rotating movement. For this, the locking body can be screwed onto the dosing unit from above.

The movement of the locking body from the closed position to the drainage position may be or comprise a rotating movement.

For this, the dosing unit and the locking body may comprise bayonet like guiding elements, which allow a linear movement of the locking body from the open to the closed position, and further allow a rotating movement of the locking body from the closed position to the drainage position. The guiding elements may have one or more guiding pins which interact with one or more guiding grooves of the other element. The guiding grooves can have a linear section and an inclined circumferential section. The angle of inclination of the circumferential section can be used to determine the force required to open the portion container.

Alternatively, the dosing unit and the locking body may comprise inter-acting threaded portions, wherein the locking body can be moved by a screw movement first from the open to the closed position and then further to the drainage position. Here, the force required to open the portion container can also be determined constructively via the slope of the threaded portions.

The movement of the locking body from the open position to the closed position and/or from the closed position to the drainage position may involve a pivoting movement. For this purpose, the locking body can be connected to the dosing unit via a joint.

According to a possible embodiment of a nebulizer, the dosing unit may comprise a removal handle for discharged portion containers.

In this regard, the removal handle may comprise retaining elements which engage the portion container during movement of the locking body from the open to the closed position and/or from the closed position to the drainage position, and retain it during movement of the locking body back to the open position. The emptied portion container can thus be removed automatically with the locking body.

The removal handle may further comprise ejection means which release the portion container from the retaining elements. This allows the emptied portion container to be easily released afterwards.

According to another possible embodiment, the removal handle may comprise a lateral recess of the uptake chamber through which a handling tongue of the portion container is or can be guided. The handling tongue can then be easily taken out of the uptake chamber after application of the nebulizer.

According to a further embodiment, the removal handle may comprise a discharge lever which engages behind the portion container on a side opposite to the opening of the uptake chamber. This allows the portion container to be lifted in the uptake chamber for easy removal.

According to a further aspect, the object is solved by a nebulizer comprising a nebulizer head and a control unit, wherein the control unit comprises at least one sensor and is arranged to activate the nebulizer head in dependence on measured values of the at least one sensor, wherein the nebulizer comprises a signal transmitter which is arranged to output a signal which is directly perceivable by the patient and which indicates an activation state of the nebulizer head, a measured value of the at least one sensor, and/or information derived therefrom.

Through the signal transmitter, the patient can receive immediate feedback about the activation state of the Nebulizer head or other information that may be relevant to therapy. The signal transmitter may comprise an acoustic signal transmitter, e.g. a loudspeaker for emitting signal tones. The signal transmitter may also include a visual signal transmitter, e.g. one or more signal lights or LEDs.

According to a possible embodiment of a nebulizer, the signal transmitter may comprise a haptic signal transmitter, which may be a vibration generator.

The vibration generator may be connected to an inner side of a housing of the nebulizer. Nebulizers are often designed with a housing that can be grasped with one hand. By connecting the vibration generator to the housing, the vibration can be perceived particularly directly by the patient. The vibration generator can be attached directly to the housing, or it can be arranged at a distance from the housing and connected to it via a coupling element.

The housing of the nebulizer can be configured in such a way that the propagation of the vibration along the housing is reduced or completely prevented. For this purpose, the housing may have an increased elasticity in the vicinity of the location to which the vibration generator is connected. This ensures that the vibration is not transmitted along the housing to areas where it is undesirable. This applies, for example, to a mouthpiece of the nebulizer, which can be touched by the patient with the lips or teeth. Here, a vibration might be perceived as unpleasant.

According to one possible embodiment, the signal transmitter can be set up to output at least two different signals. This allows more information to be provided to the patient.

For example, the vibration generator can be configured to emit vibration signals with different frequencies and/or intensities.

According to a further aspect, the object is solved by a nebulizer system comprising a base unit and at least one nebulizer head and/or at least one dosing unit which can be combined with the base unit to form a ready to use nebulizer, wherein the at least one dosing unit, a portion container to be inserted into the dosing unit, and/or the at least one nebulizer head are equipped with an identification feature, wherein the base unit comprises a control unit, which is configured to register the identification feature of a nebulizer head, a dosing unit, and/or a portion container, wherein further the control unit is adapted to determine by means of the identification feature whether the operation of the nebulizer head, the dosing unit, and/or the portion container with the base unit is authorized, and wherein the nebulizer head is a mesh nebulizer.

The control unit can determine whether the nebulizer system is to be operated with inadmissible components by means of the corresponding configuration.

If the control unit is further configured to activate the nebulizer head and/or the dosing unit only when these are authorized to be used with the base unit, it can be ensured that the operational safety of the nebulizer system is not compromised by non-system components. Non-system components can be counterfeit products or products from other manufacturers whose use in the nebulizer system is not authorized or not desired.

The control unit may contain a list of permitted identification features. If necessary, this list can be modified via a data connection in order to allow new components for use or to prevent the use of previously allowed components in the future.

The identification feature may comprise an RFID chip, and the control unit may comprise an RFID reader. With this configuration, the registration of the identification feature can be done easily and contactless.

The identification feature may comprise a memory chip, and the base unit and the nebulizer head, the dosing unit, and/or the portion container may be configured such that when the base unit is combined with the nebulizer head, the dosing unit, and/or the portion container, a galvanic connection is provided between the control unit and the memory chip. For this purpose, the base unit and the nebulizer head or the dosing unit may have plug-in or spring contacts by means of which the connection is established.

According to a possible embodiment, the identification feature may include information about a durability and/or a maximum period of use of the nebulizer head, the dosing unit, and/or the portion container. In addition to the basic suitability of the nebulizer head or the dosing unit and/or the portion container, the control unit may also check whether they have exceeded a durability and/or period of use and can therefore no longer be used safely.

The control unit may be configured to write usage data back to the RFID chip or the memory chip. In this way, compliance with a predefined maximum number of uses and/or a maximum period of use after the first use can also be monitored by the control unit.

The object is also solved by a nebulizer head of a nebulizer system, wherein the nebulizer head is equipped with an identification feature.

The object is further solved by a dosing unit and/or by a portion container of a nebulizer system, wherein the dosing unit and/or the portion container are equipped with an identification feature.

The object is also solved by a base unit of a corresponding nebulizer system.

The invention is described in more detail below with reference to a number of exemplary figures, whereby the embodiments shown in are merely intended to contribute to a better understanding of the invention without limiting it.

It is shown:

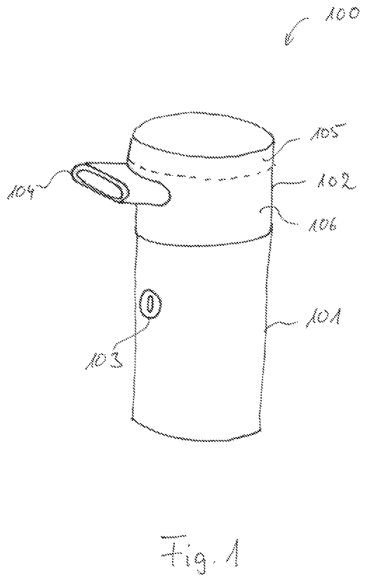

FIG. 1: a nebulizer,

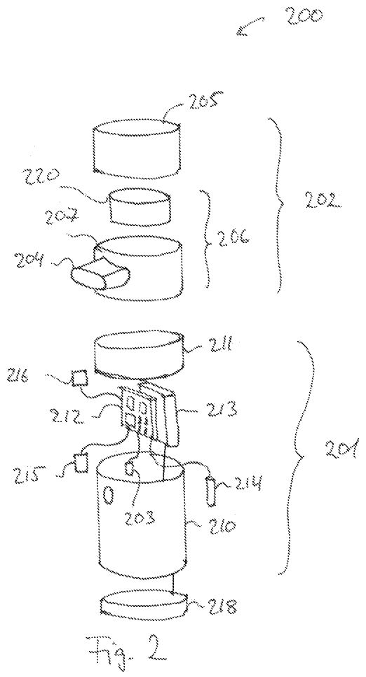

FIG. 2: An exploded view of a nebulizer,

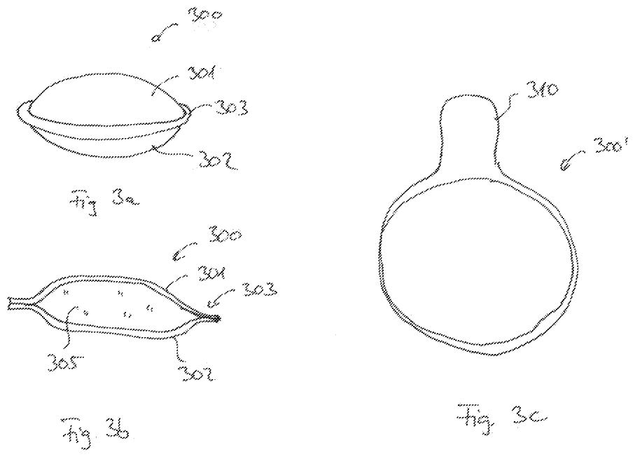

FIG. 3a-c: portion container,

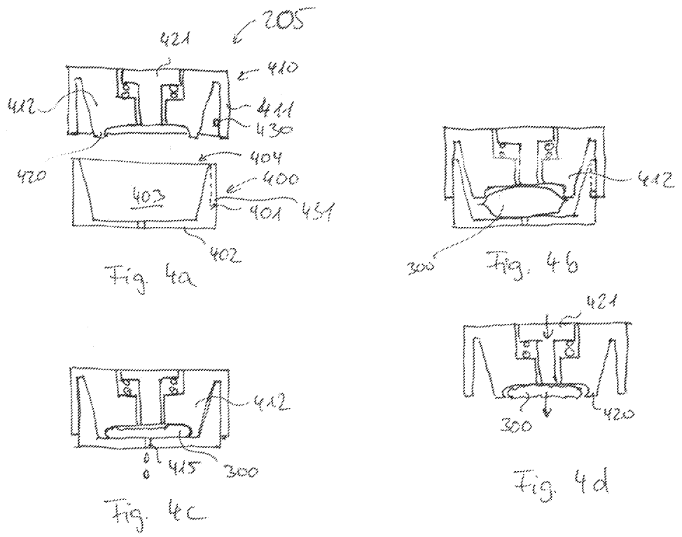

FIG. 4a-d: a dosing unit,

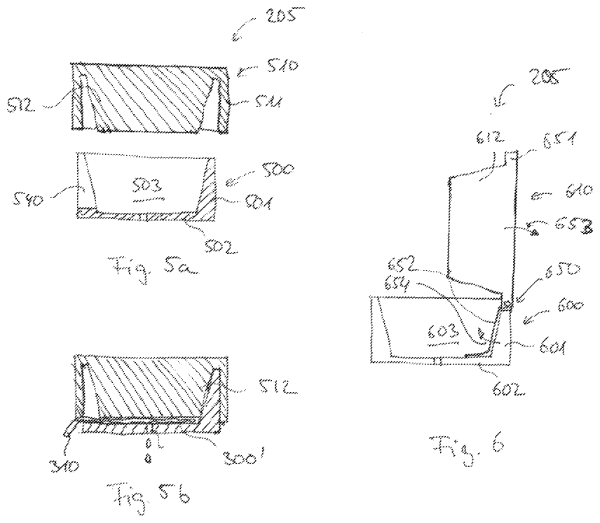

FIG. 5a, b: a further dosing unit,

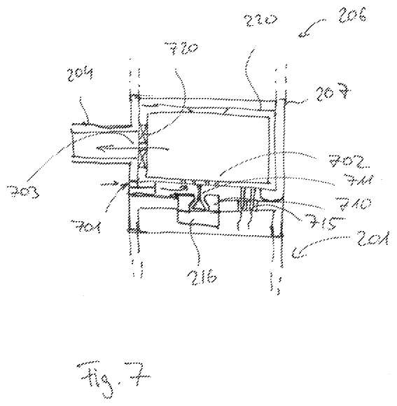

FIG. 6: yet a further dosing unit,

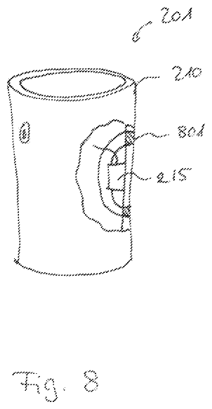

FIG. 7: a nebulizer head,

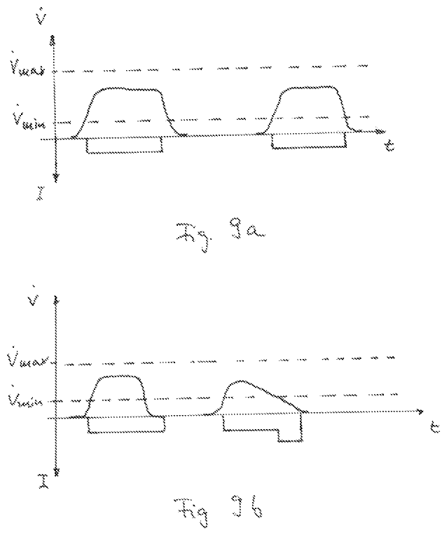

FIG. 8: a base of a nebulizer,

FIG. 9a, b: Activation sequences of a vibration generator.

FIG. 1 shows a nebulizer 100. The nebulizer 100 comprises a base 101 and a head 102. The base 101 and the head 102 of the nebulizer have a substantially circular cylindrical contour which can be easily handled with one hand.

A main switch 103 is arranged at the base 101 of the nebulizer 100, with which the nebulizer 100 can be switched on and off. A mouthpiece 104 is arranged on the head 102 of the nebulizer 100.

The head 102 of the nebulizer 100 comprises a dosing unit 105 and the actual nebulizer head 106, which contains an aerosol generator. In the example shown, the dosing unit 105 and the nebulizer head 106 are shown as a single assembly, but they can also be designed as separate assemblies.

FIG. 2 shows an exploded view of a nebulizer 200. The nebulizer 200 again comprises a base 201 and a head 202.

The base 201 of the nebulizer 200 comprises a lower housing part 210 and an upper housing part 211. A control unit 212 and an energy storage 213 are accommodated in the lower housing part 210. The energy storage 213 can be an accumulator, for example a Li-ion accumulator.

The control unit 212 is connected to the main switch 203 and can be switched on and off. Furthermore, the control unit 212 is connected to an antenna assembly 214, a vibration generator 215, and a flow sensor 216. The antenna assembly 214 is used to establish a communication link, for example a Bluetooth, mobile phone or WiFi connection. The function of the other components will be explained later.

The base 201 may further comprise a charging assembly 218 for contactless charging of the energy storage 213. The charging assembly 218 may terminate the lower housing part 210 at the bottom.

The upper housing part 211 seals the base 201 so that the electronic components inside are protected from moisture. At the same time, the upper housing part 211 provides a connection to the head 202 of the nebulizer 200.

The head 202 of the nebulizer 200 comprises a metering assembly 205 and a nebulizer head 206. The nebulizer head 206 comprises a head housing 207, on which a mouth piece 204 is arranged, and an aerosol generator 220 arranged in the head housing 207.

Although the mouth piece 204 is shown as being integral with the head housing 207, the mouth piece 204 may also be a separate component which is connected to the head housing 207 by suitable connecting means. For example, the head housing 207 can have a nozzle onto which the mouth piece 204 is attached.

The dosing unit 205 is used to deliver a defined amount of a therapeutic agent to the aerosol generator 220. In contrast to known dosing units, which require manual dosing of the desired amount of therapeutic agent, the dosing unit 205 is set up to receive and process pre-filled portion containers with the therapeutic agent. Possible embodiments of such portion containers are shown in FIGS. 3a to 3c.

FIG. 3a shows a portion container 300 in a perspective view, FIG. 3b shows the same portion container 300 in a sectional view. The portion container 300 comprises a top cover 301 and a bottom cover 302, which are connected along a sealing seam 303. A volume 305 is formed between the top cover 301 and the bottom cover 302, which is filled with the therapeutic agent.

The top cover 301 and the bottom cover 302 may, for example, consist of a metal foil. Alternatively, the top and bottom covers 301, 302 may be made of a plastic film, for example a thermoplastic polymer. Examples of such polymers are polyethylene (PE), polyethylene terephthalate (PET), or polyetheretherketone (PEEK).

The sealing seam 303 may be produced, for example, by gluing, pressing or welding the top cover 301 and the bottom cover 302.

FIG. 3c shows an alternative version of a portion container 300′. Unlike the portion container 300, the portion container 300 has a handling tongue 310, the function of which will be explained later.

FIGS. 4a-d show a possible configuration of the dosing unit 205. The dosing unit 205 comprises a receiving body 400 with a circumferential wall 401 and a base 402. Wall 401 and base 402 form an uptake chamber 403 with an upwardly facing opening 404, through which a portion container not shown can be inserted into the uptake chamber 403.

The dosing unit 205 further comprises a locking body 410 in the form of a lid, which can be placed on the receiving body 400 to close the opening 404. For this purpose, the locking body 410 has a collar 411 which encloses the wall 401.

The locking body 410 further comprises a displacer body 412 configured to be immersed in the uptake chamber 403 when the locking body 410 is placed on the receiving body 400.

FIG. 4a shows the dosing unit 205 in an open position. FIG. 4b shows the dosing unit in a closed position with the portion container 300 inserted. In the closed position, the displacer body 412 rests against the portion container.

FIG. 4c shows the dosing unit 305 in a drainage position. In this position, the Displacer body 412 enters deeper into the Uptake chamber 403 and presses on the Portion container 300, causing it to tear open and empty. The point at which the portion container 300 tears open can be predetermined by the design. For this purpose, the top and/or bottom cover 301, 302 may have tear lines which are not shown. The therapeutic agent can then drain through a dosing opening 415 and enter the nebulizer head, which is not shown.

In order to remove the emptied portion container from the uptake chamber 403, the locking body 410 comprises a removal handle in the form of gripping hooks 420 which grip the portion container 300 when the locking body 410 is moved into the drainage position.

When the locking body 410 is then moved back to the open position, it takes the emptied portion container 300 with it, as shown in FIG. 4d.

To release the emptied portion container 300 from the gripper hooks 420, the locking body 410 has a spring-loaded ejection plunger 421. This can be moved by pressing on the top of the ejection plunger to push the portion container 300 out of the gripper hooks 420.

In order to move the locking body 410 from the open position to the closed position and to the drainage position, a bayonet-like guide may be provided. For this purpose, a pin 430 is arranged on the collar 411, which runs in a guiding groove 431 in the wall 401. The guiding groove 431 extends straight down from the upper edge of the wall 401 to a depth corresponding to the closed position. Further, the guiding groove 431 then runs at an angle so that the locking body 410 can be moved in a screwing motion into the drainage position. The pitch of the guiding groove 431 in this section can be used to determine the force required to empty the portion container 300.

FIGS. 5a, 5b show an alternative embodiment of the dosing unit 205, whereby similar components are marked with a reference sign increased by 100. The wall 501 of the receiving body 500 has a lateral recess 540 through which a handling tongue 310 of the portion container 300′ can be guided. This handling tongue 310 can be used to remove the emptied portion container 300′ from the uptake chamber 503.

FIG. 6 shows another possible embodiment of the dosing unit 205 in which the locking body 610 can be moved in a pivoting movement between the open, the closed and the drainage position. For this purpose, the locking body is attached to the receiving body with a joint 650.

In the embodiment shown in FIG. 6, the locking body 610 does not have a collar, but only the displacer body 612 and a stop rim 651.

As a removal handle for emptied portion containers, the dosing unit 205 in this embodiment has a discharge lever 652, which lifts the portion container not shown. For this purpose, the discharge lever 652 can be coupled to the locking body 612 in such a way that it is automatically raised beyond the open position by a pivoting movement of the locking body 610 (arrows 653, 654).

FIG. 7 shows a schematic representation of a nebulizer head 206. The nebulizer head 206 comprises a head housing 207 with mouth piece 204, as well as an aerosol generator 220. The internal structure of the aerosol generator 220 is not shown for reasons of clearness; it can be an ultra sonic nebulizer or a mesh nebulizer, for example.

The air flow in the nebulizer head 206 is as follows: Air enters the nebulizer head 206 through intake openings 701 in the area of the mouth piece 204, and then enters the aerosol generator 220 centrally from below through inlet openings 702. After mixing with the aerosol, the air enters the mouth piece 204 through an outlet opening 703 of the aerosol generator 220 and is then inhaled by the patient.

The intake openings 701 are arranged in the area of the mouth piece 204 to prevent accidental blocking of the intake openings 701 when using the nebulizer.

Since the air flow is driven by a negative pressure generated by the patient, a negative pressure is also created under the aerosol generator 220, which is approximately proportional to the volume flow of the inhaled air. This negative pressure is measured by the flow sensor 216, which is located below the nebulizer head 206 in the base 201.

A valve 710 is arranged between the nebulizer head 206 and the sensor 216. The valve 710 is opened by a pin 711, which is located centrally under the aerosol generator 220. The valve 710 prevents contamination of the sensor 216 when the nebulizer head 206 is not placed on the base 201.

An additional valve, not shown, may be provided to prevent airflow through the nebulizer head 206 in the opposite direction to that intended, for example if a user inadvertently blows into the mouth piece 204. The additional valve is preferably a one-way valve, i.e. a valve that allows airflow in only one direction, for example a lip valve.

Plug-in contacts 715 of the aerosol generator 220 connect it to the control unit not shown in FIG. 7; they serve to activate and supply the aerosol generator 220.

The aerosol generator 720 carries an RFID chip 720, which in the illustrated embodiment is arranged in a ring shape around the outlet opening 703, whereby the shape of the RFID chip 720 is mainly determined by its antenna. The RFID chip 720 may also be arranged at other locations of the aerosol generator 220. The RFID chip may be encased in silicone to protect it from moisture.

Instead of the RFID chip 720, the aerosol generator 220 may also comprise a galvanically coupled memory chip. This may, for example, be connected to the control unit via the plug-in contacts 715.

The RFID chip 720 or the memory chip contains identification features of the aerosol generator 220 or the nebulizer head 206. These can be read by the control unit to determine whether the nebulizer head 206 is compatible with the base 201 and may be used. Further, the RFID chip or memory chip may contain data about a durability and/or maximum period of use, as well as the number of uses already performed. This data can also be stored on the chip by the control unit after use.

The control unit also uses this data to check the permissibility of using or continuing to use the nebulizer head with the base 201. In the event of a negative result, the control unit can refrain from activating the nebulizer head and, if necessary, issue a malfunction message.

FIG. 8 shows a section of the lower housing part 210 of the base 201 of a nebulizer, with the housing wall shown in partial section.

The vibration generator 215 is attached to the housing wall at a position on the housing part 210 that is approximately opposite the mouth piece, which is not shown. The position is chosen so that a vibration generated by the vibration generator 215 is easily perceived by a user of the nebulizer.

In order to prevent the vibration from spreading to areas of the nebulizer where vibration is undesirable, the mounting point of the vibration generator 215 is surrounded by a damping material layer 801, which does not transmit the vibrations or only dampens them.

Areas where vibration is undesirable include the mouth piece in particular.

The damping material layer preferably has a higher elasticity than the surrounding material, it can for example be made of silicone or EPDM rubber.

The vibration generator 215 is used to provide feedback to a user of the Nebulizer about its use.

The control unit 212 activates the nebulizer head 206 depending on the respiratory flow measured via the sensor 216. However, since the user cannot directly perceive this activation, the correct application is difficult for the user to control.

To improve this, the control unit 212 may activate the vibration generator at the same time as the nebulizer head 206, so that the user receives direct feedback.

In addition to the direct feedback about the activation, the control unit 212 may also provide further information to the user by means of the vibration generator 215.

For example, if a user's breath duration does not correspond to a desired activation duration of the nebulizer head 206, the control unit 212 may continue to activate the vibration generator 215 after the breath is completed to communicate to the user that he or she should inhale longer. The duration of further activation by the control unit 212 may be dynamically adjusted to gradually bring the user to the desired breath duration without causing frustration. Likewise, a dynamic adjustment can be made to slowly bring the user to his physiologically determined maximum breath length.

The control unit 212 may further provide feedback to the user if the breath is too weak or too strong. To do so, the vibration generator 215 can be activated with a different frequency and/or intensity.

Possible activation patterns of the vibration generator are shown in FIGS. 9a, b.

FIG. 9a shows a diagram with two breaths. The time is plotted on the longitudinal axis and the volume flow V of the inhaled air is plotted on the upper vertical axis, both in arbitrary units. A maximum volume flow V′max and a minimum volume flow V′min, which are necessary or authorized for the correct functioning of the aerosol generator 220, are also entered.

The activation state I of the vibration generator 215 is displayed on the lower vertical axis (below the longitudinal axis). For both breaths, the vibration generator 215 is activated when the flow rate exceeds V′min and deactivated after the aerosol generator 220 has reached a preset activation time.

FIG. 9b shows a similar diagram with two breaths. The first breath is shorter than the intended activation time of the aerosol generator 220, so the vibration generator 215 continues to be activated even after the breath has ended.

The second breath in the diagram of FIG. 9b shows a strongly decreasing volume flow at the end. This is signaled to the user by the fact that the vibration generator 215 is activated with a changed intensity as soon as the volume flow falls below the minimum V′min.

Referring back to FIG. 2, it can be seen that the entire nebulizer 200 has a highly modular design and can thus be easily varied in terms of construction. For example, instead of the closed dosing unit 205, a manual dosing of a therapeutic agent into the aerosol generator 220 can be provided; for this purpose, the dosing unit 205 would be replaced by a removable lid, not shown, which closes the nebulizer head 206.

The nebulizer head 206 is additionally configured in such a way that the aerosol generator 220 can be removed for cleaning purposes and/or replaced when worn. At the same time, the aerosol generator 220 is configured as a rotationally symmetrical component, which may also be easily integrated into other systems, e.g. into stationary inhalation or respiration devices.

The embodiments described above serve only to explain various aspects of the invention. The individual aspects described can also solve some of the objects of the invention and achieve the advantages mentioned without implementing other aspects. In particular, the described embodiments of the dosing unit, the nebulizer head, and the base can be combined with each other and/or with components from the prior art to obtain a functional and advantageous nebulizer.

1. A mesh nebulizer comprising a dosing unit for providing a predetermined amount of a therapeutic agent, wherein the therapeutic agent is delivered in a portion container to be opened by pressure;
wherein the dosing unit comprises an uptake chamber with a dosing opening for dispensing the therapeutic agent, an opening for inserting a portion container and a locking body for the opening;
wherein the locking body is adapted to be moved from an open position in which the locking body releases the opening to a closed position in which the locking body closes the opening; and
wherein the locking body is further adapted to be moved from the closed position to a drainage position in which the locking body applies pressure to the portion container to open the portion container and release the therapeutic agent.

2. The mesh nebulizer according to claim 1, characterized in that the movement of the locking body from the open position to the closed position is or comprises a linear movement.

3. The mesh nebulizer according to claim 1, characterized in that the movement of the locking body from the open position to the closed position is or comprises a rotating movement.

4. The mesh nebulizer according to claim 1, characterized in that the movement of the locking body from the closed position to the drainage position is or comprises a rotating movement.

5. The mesh nebulizer according to claim 4, characterized in that the dosing unit and the locking body have bayonet-like guiding elements.

6. The mesh nebulizer according to claim 4, characterized in that the dosing unit and the locking body have inter-acting threaded portions.

7. The mesh nebulizer according to claim 1, characterized in that the movement of the locking body from the open position to the closed position and/or from the closed position to the drainage position involves a pivoting movement.

8. The mesh nebulizer according to claim 1, characterized in that the dosing unit comprises a removal handle for discharged portion containers.

9. The mesh nebulizer according to claim 8, characterized in that the Removal handle comprises retaining elements which engage the portion container during movement of the Locking body from the open to the closed position and/or from the closed position to the drainage position, and retain it during movement of the locking body back to the open position.

10. The mesh nebulizer according to claim 9, characterized in that the removal handle comprises ejection means which release the portion container from the retaining elements.

11. The mesh nebulizer according to claim 8, characterized in that the removal handle comprises a lateral recess of the uptake chamber through which a handling tongue of the portion container is or can be guided.

12. The mesh nebulizer according to claim 8, characterized in that the removal handle comprises a discharge lever which engages behind the portion container on a side opposite to the opening of the uptake chamber.

13. A nebulizer system for a mesh nebulizer comprising a base unit and at least one nebulizer head and/or at least one dosing unit which can be combined with the base unit to form a ready to use nebulizer,
wherein the at least one dosing unit, a portion container to be inserted into the dosing unit, and/or the at least one nebulizer head are equipped with an identification feature,
wherein the base unit comprises a control unit, which is configured to register the identification feature of a nebulizer head, a dosing unit, and/or a portion container,
wherein further the control unit is adapted to determine by means of the identification feature whether the operation of the nebulizer head, the dosing unit, and/or the portion container with the base unit is authorized,
and wherein the nebulizer head is a mesh nebulizer.

14. The nebulizer system according to claim 13, characterized in that the control unit is configured to activate the nebulizer head and/or the dosing unit only when these are authorized to be used with the base unit.

15. The nebulizer system according to claim 13, characterized in that the identification feature comprises an RFID chip, and that the control unit comprises an RFID reader.

16. The nebulizer system according to claim 13, characterized in that the identification feature comprises a memory chip, and that the base unit and the nebulizer head, the dosing unit, and/or the portion container are configured such that when the base unit is combined with the nebulizer head, the dosing unit, and/or the portion container, a galvanic connection is provided between the control unit and the memory chip.

17. The nebulizer system according to claim 13, characterized in that the identification feature includes information about a durability and/or a maximum period of use of the nebulizer head, the dosing unit, and/or the portion container.

18. The nebulizer system according to claim 17, characterized in that the control unit is configured to write usage data back to the RFID chip or the memory chip.

19. A nebulizer head for a nebulizer system according to claim 13, characterized in that the nebulizer head is equipped with an identification feature.

20. A dosing unit for a nebulizer system according to claim 13, characterized in that the dosing unit is equipped with an identification feature.

21. A portion container for a nebulizer system according to claim 13, characterized in that the portion container is equipped with an identification feature.

22. Base unit for a nebulizer system according to claim 13.
